(19) United States
(12) Patent Application Publication
(10) Pub. No.: US 2017/0121129 A1
(43) Pub. Date: May 4, 2017

(54) ROBOTIC CONTROL DEVICE

(71) Applicant: IMPERIAL INNOVATIONS LIMITED, Great London (GB)

(72) Inventors: George MYLONAS, Greater London (GB); Guang-Zhong YANG, Greater London (GB)

(73) Assignee: IMPERIAL INNOVATIONS LIMITED, Greater London (GB)

(21) Appl. No.: 13/993,139

(22) PCT Filed: Dec. 13, 2011
(86) PCT No.: PCT/GB11/01714

§ 371 Date: Aug. 7, 2013

(30) Foreign Application Priority Data
Dec. 13, 2010 (GB) 1021124.1

(51) Int. Cl. B65G 51/03; A61B 90/60; A61B 34/00; B25J 9/16; B25J 13/02

(52) U.S. Cl. CPC B65G 51/03; B25J 9/1689; B25J 13/025; A61B 34/76; A61B 34/74; A61B 90/60; A61B 34/35

(57) ABSTRACT

A robotic control device for receiving manipulation instructions for an end effector of a surgical instrument has a platform for supporting a haptic manipulator device, the platform being movably coupled to a surface for supporting the platform and having a movement facilitating member operable to change the amount of force required to move the platform relative to the surface.

This invention relates to a control device for robots and in particular, but without limitation, an improved control device for receiving manipulation instructions for an end effector of a surgical instrument.

Robotic manipulators provide a way for humans to interface with a computer so as to provide the computer with instructions for manipulating an end effector. By allowing an operator to provide instructions via a robotic manipulator, the end effector may be controlled by the operator in a manner that is sympathetic with, or follows, the movement that the operator would perform if they were holding and manipulating the end effector themselves. Manipulators may be provided with haptic feedback components to provide the operator with an indication of forces encountered by the end effector.

One exemplary field in which robotic manipulators may be employed is that of surgery, for example telesurgery. When employed for surgery the robotic manipulator may be for controlling an end effector, such as a scalpel, forceps, or a manipulating member, by a surgeon who may not be in the same operating theatre, or even the same country, as the patient.

A person skilled in the art will appreciate that, whilst the following is described with reference to a surgical robot, the subject matter described herein may equally be applied to robots in other fields of technology. Non limiting examples of such other fields of technology include the control of robots: for sensitive and/or dangerous chemical procedures, for bomb disposal, for deep sea exploration (for example, using remotely operated underwater vehicles (ROVs)), and for teleoperated robot for space missions, for which long manipulation sessions are involved.

According to an aspect of the present invention, there is provided an apparatus having one or more surfaces that are arranged to support a robotic device for receiving instructions for an end effector. The robotic device is preferably a haptic manipulator device, for example a bespoke designed master manipulators or a generic haptic device such as a PHANTOM OMNI device as produced by SensAble Technologies, Inc. of Massachusetts, United States. The one or more surfaces form a platform for the haptic manipulator and are rigidly coupled to an operator engagement member. The operator engagement member may take the form of an arm, wrist, or hand rest, and/or may be arranged to be physically coupled to an operator—for example so as to form an exo-skeleton of sorts. The operator engagement member is arranged to be coupled (either directly or indirectly) to a fixed body, such as a work surface or desk, by a linkage that is arranged to allow the operator engagement member to move with respect to the fixed body in one or more degrees of freedom. An example linkage may have a clamp for fixedly coupling a first portion of the linkage to the fixed body and a second portion fixedly coupled to the operator engagement member. The first and second portions of the linkage may be movable relative to one another by way of one or more pivots, each of which may have an associated rotational encoder to enable determination of the spatial location of the arm rest. The spatial location of the arm rest and/or movements thereof may correspond to specific instructions provided by the operator for the movement of the end effector and/or other operations that the operator may wish for the system to perform—for example a change in the intensity of lighting in the operating theatre. The platform has a movement facilitating portion, for example a friction reducing portion such as an air cushion forming device. A person skilled in the art will recognise other friction reducing devices that could alternatively be employed with the subject matter described herein, for example: buoyancy based platforms, fluid bearings, magnetic levitation (maglev), superconducting magnetic bearings, low friction coatings, and/or carbon films. The movement facilitating portion is preferably arranged so as to reduce friction between the platform and the fixed body so as to facilitate movement of the operator engagement member, and therefore the haptic manipulator device, relative to the fixed body.

As one possibility, the apparatus, and preferably the movement facilitating portion, may be arranged so as to be able to impede movement between the platform and the fixed body, for example by the provision of a suction force.

Embodiments of the present invention will now be described, by way of example, with reference to the accompanying drawings, in which:

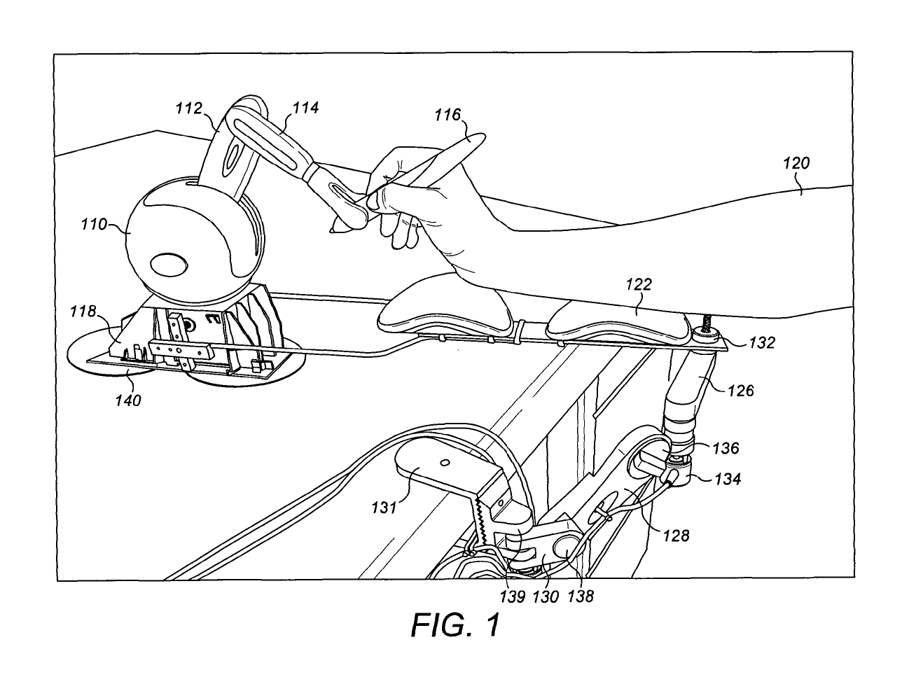

FIG. 1 shows an example embodiment of the robotic manipulator; and

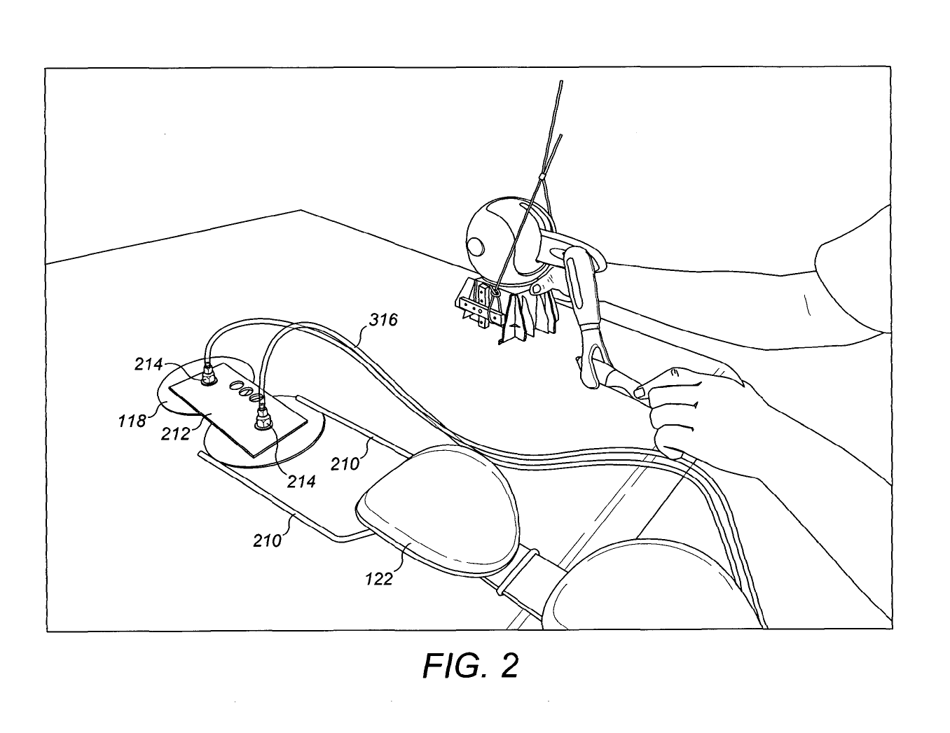

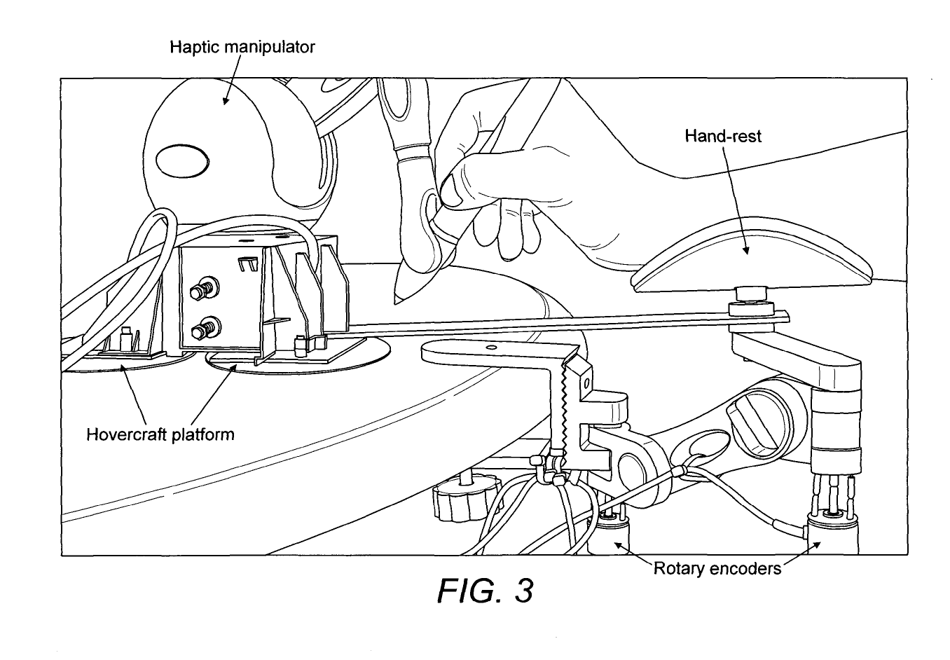

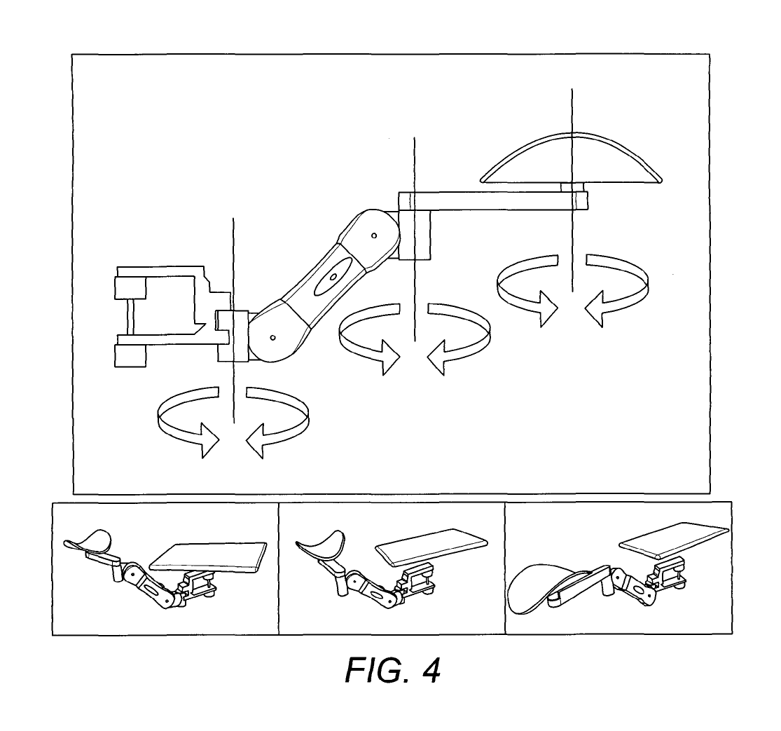

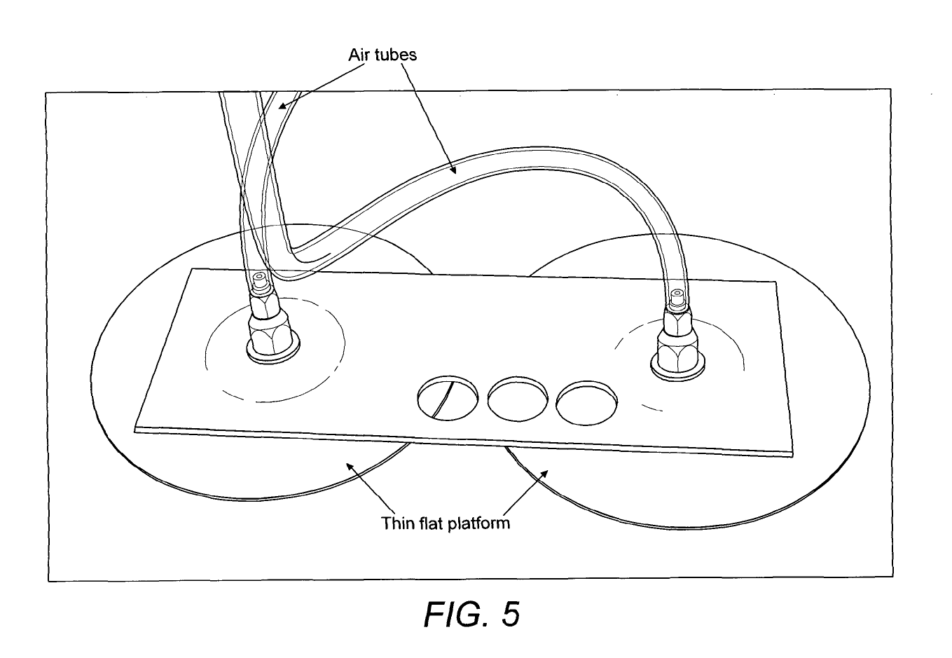

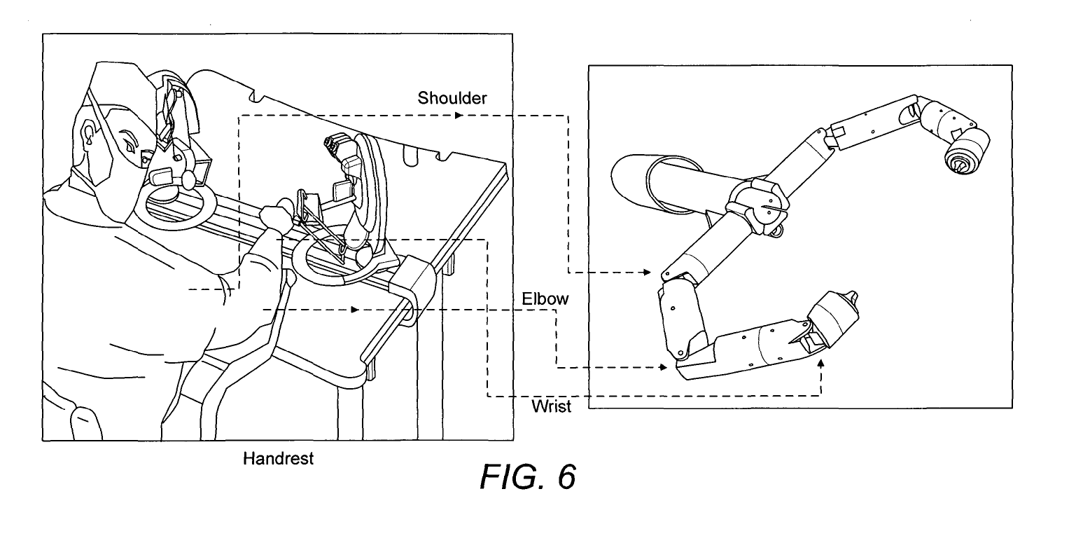

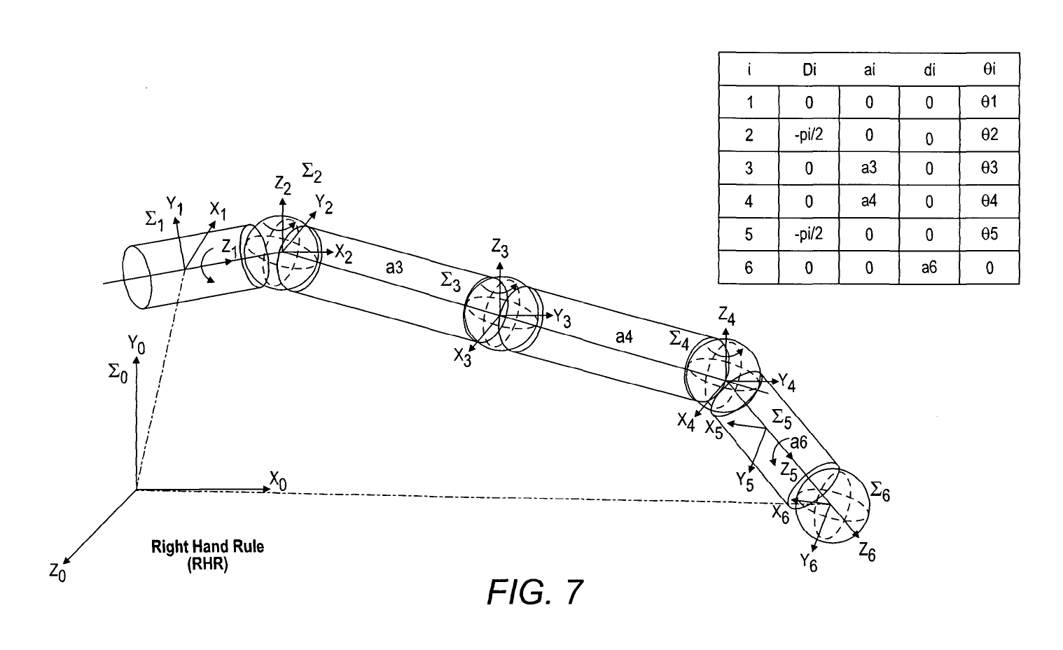

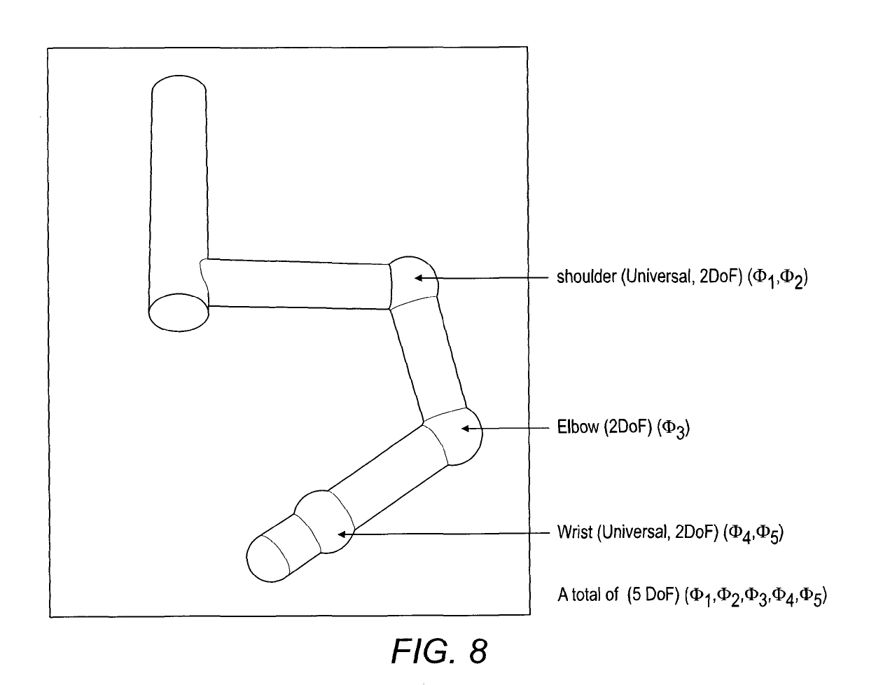

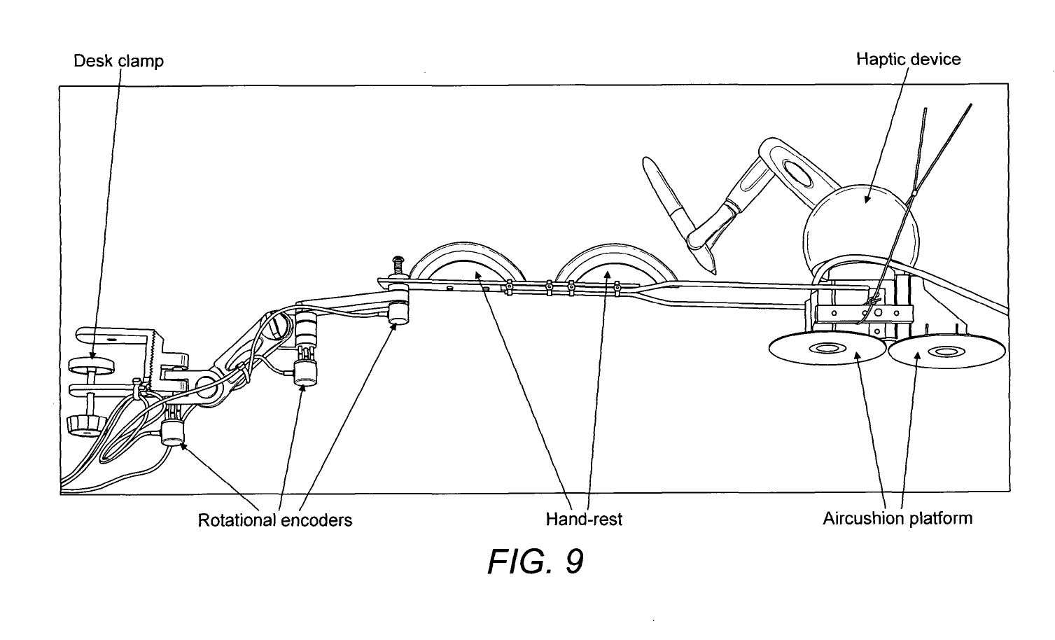

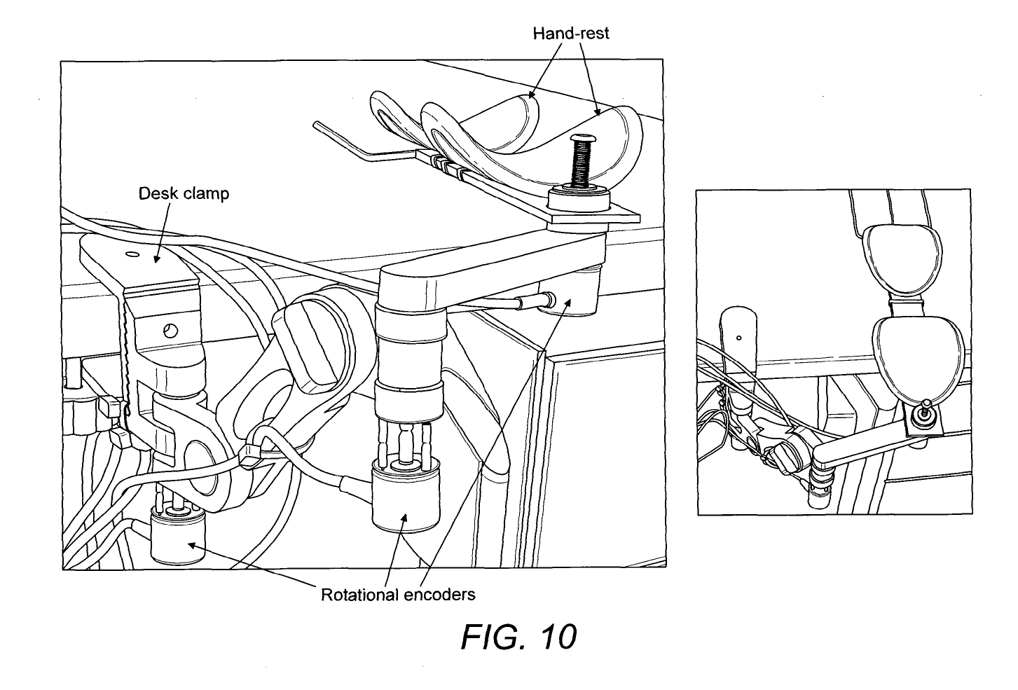

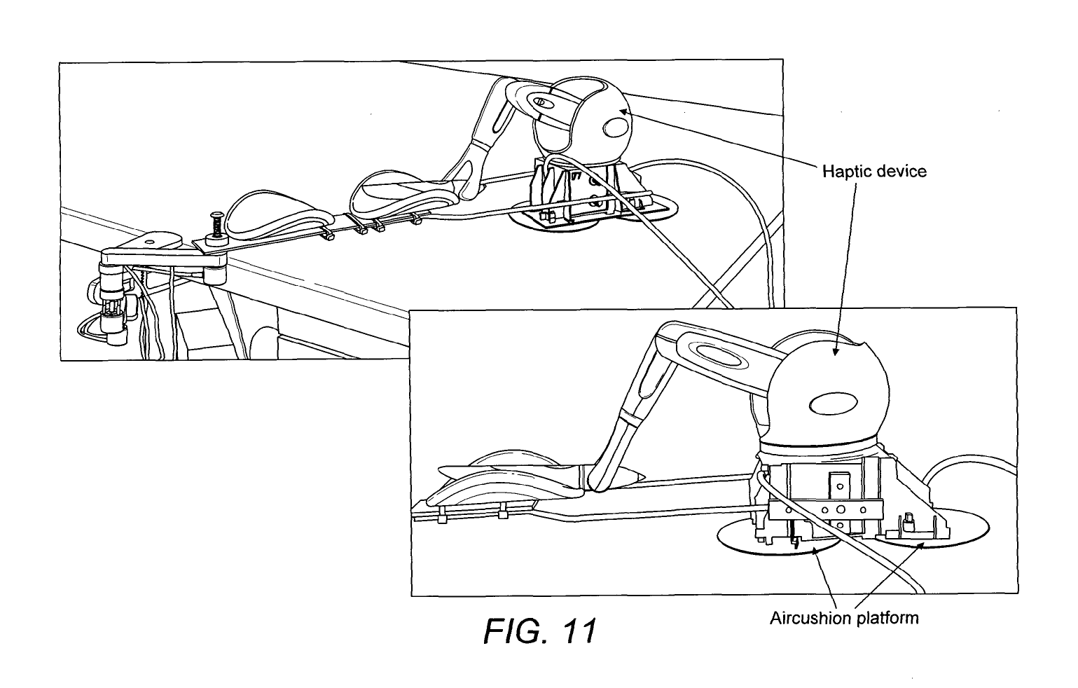

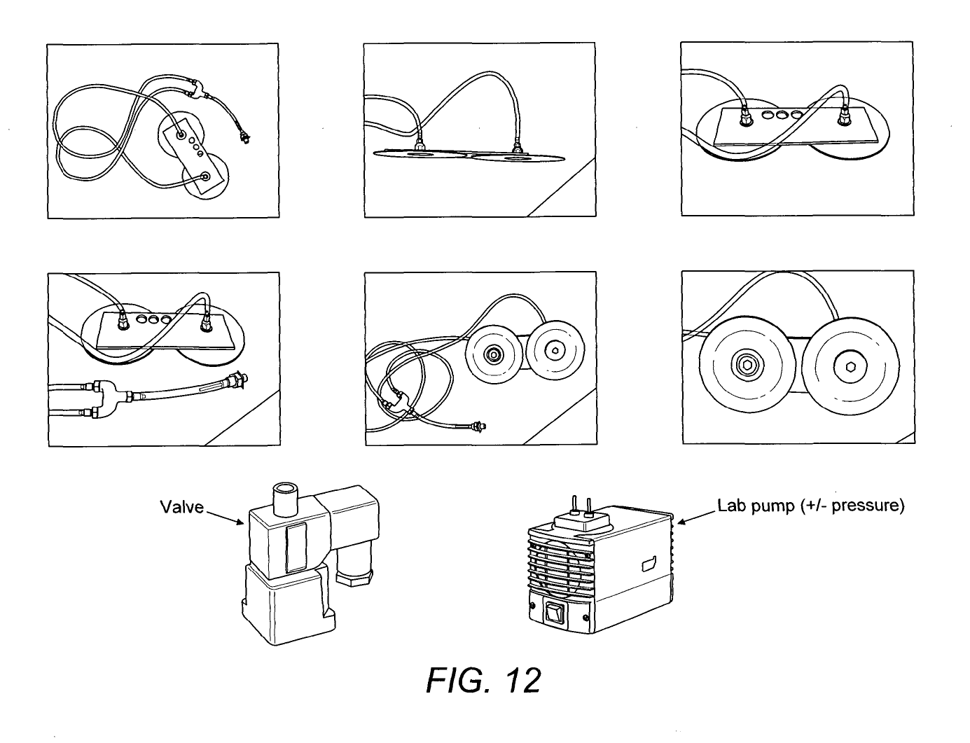

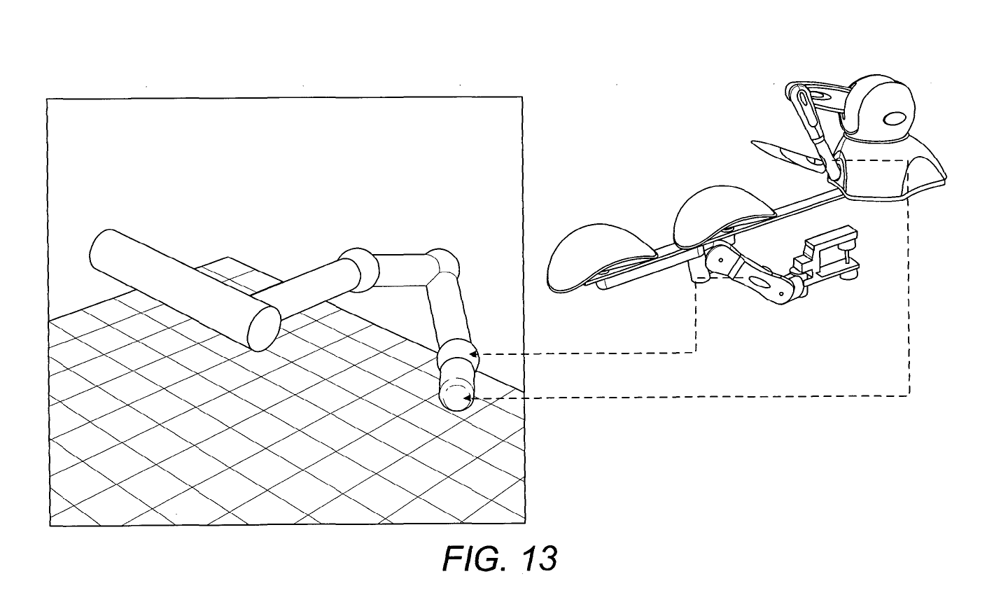

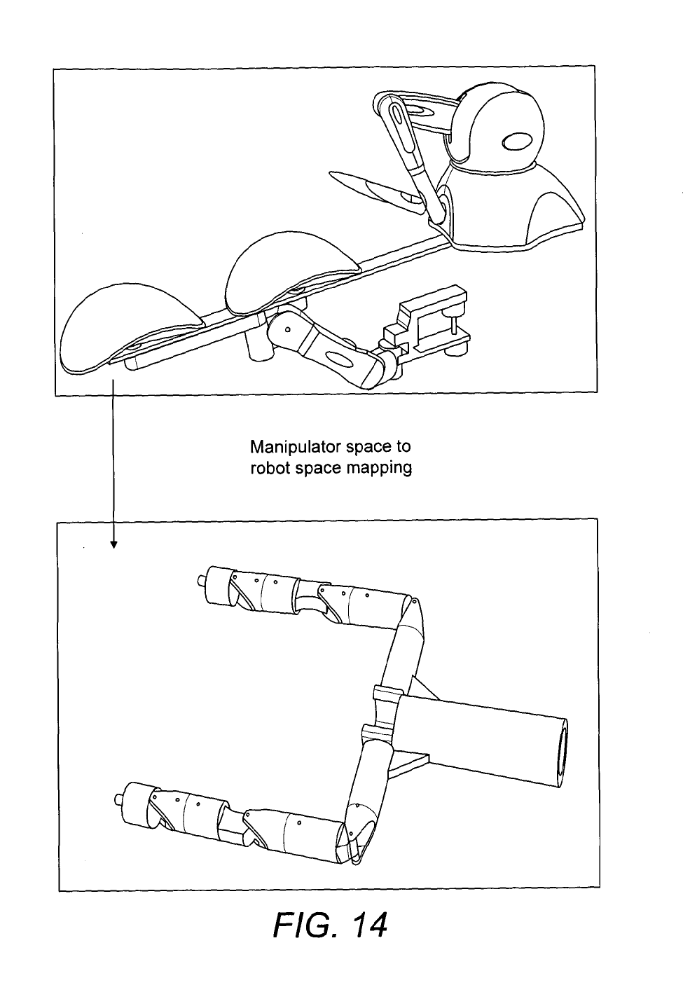

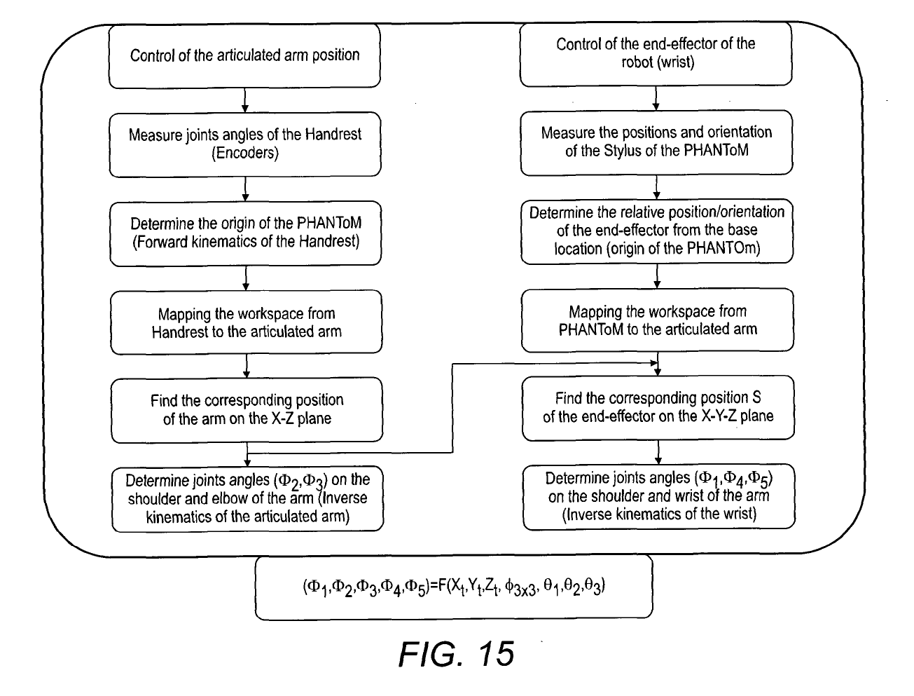

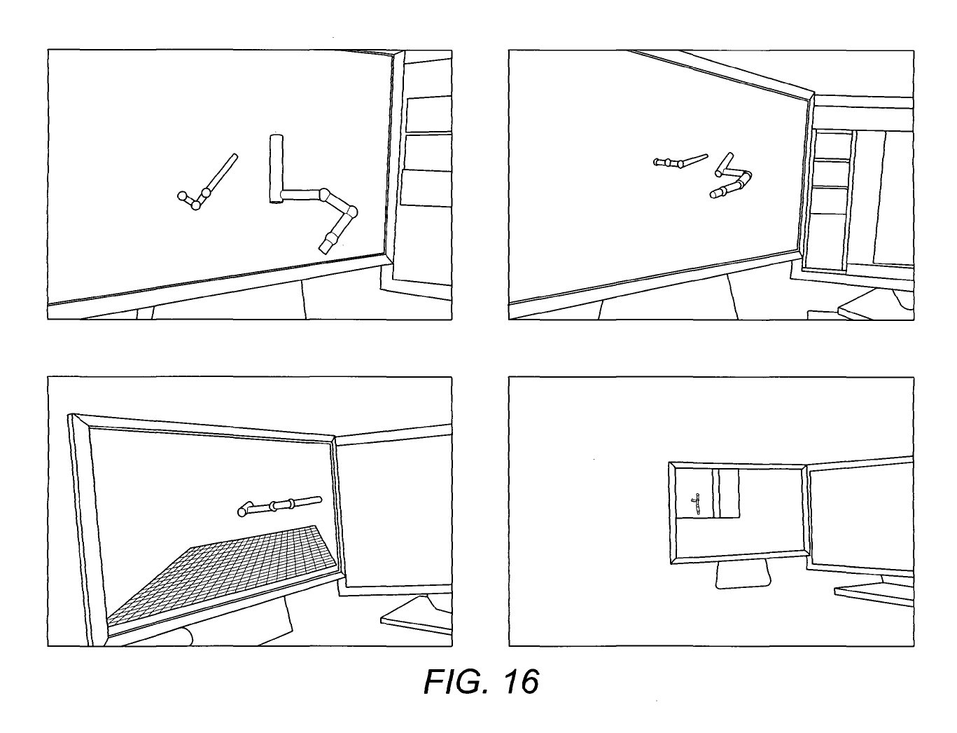

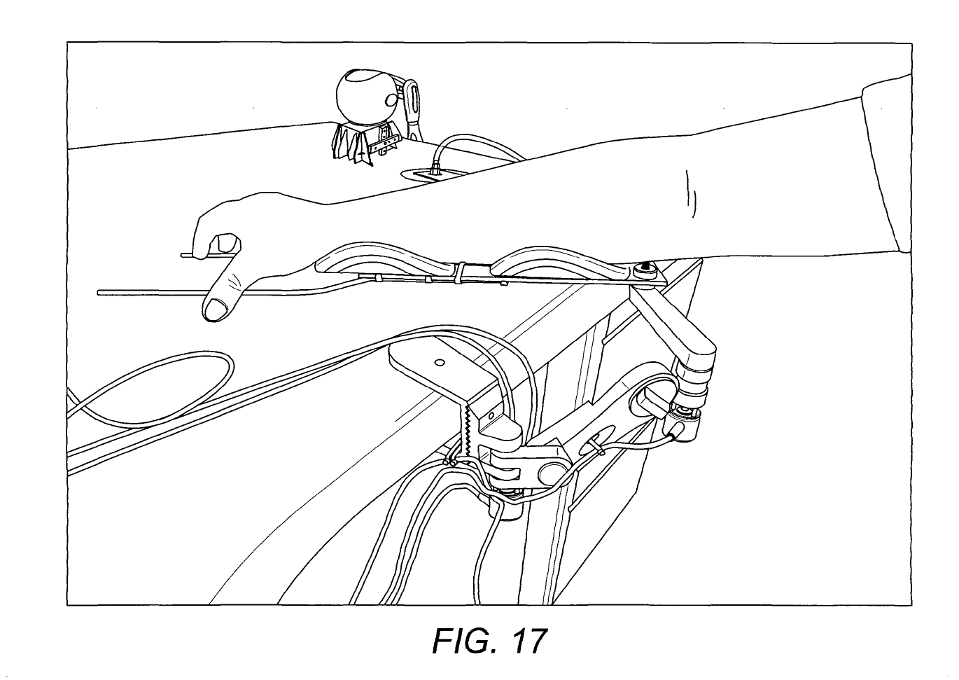

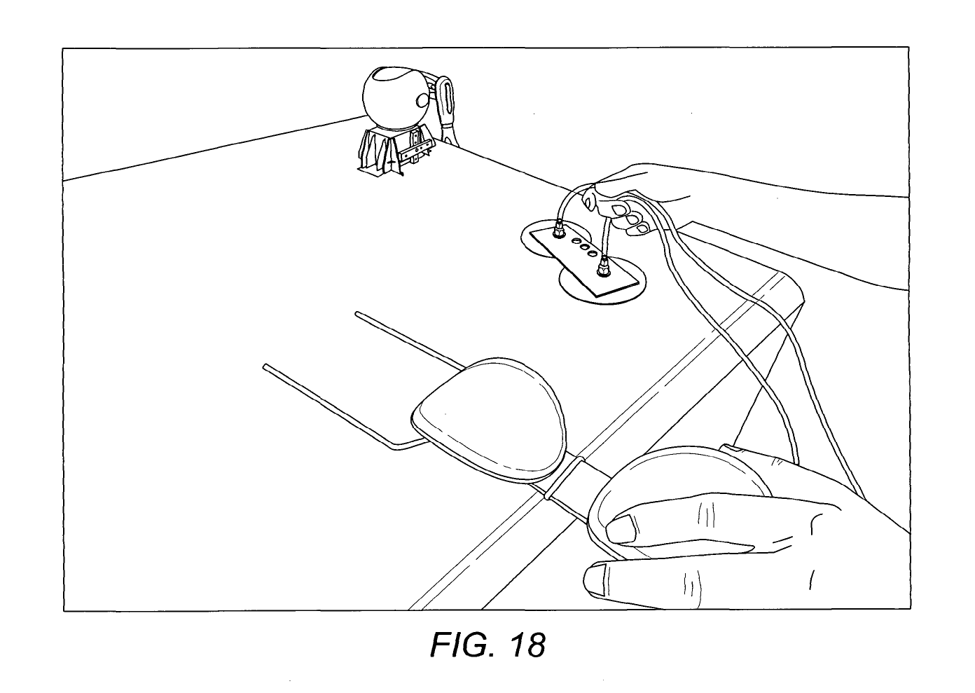

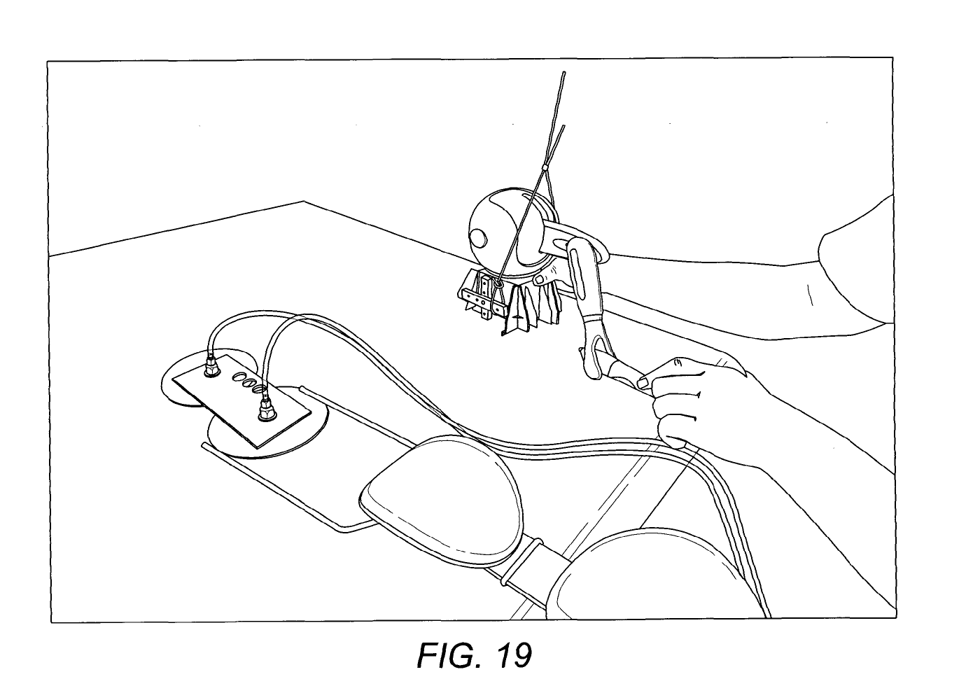

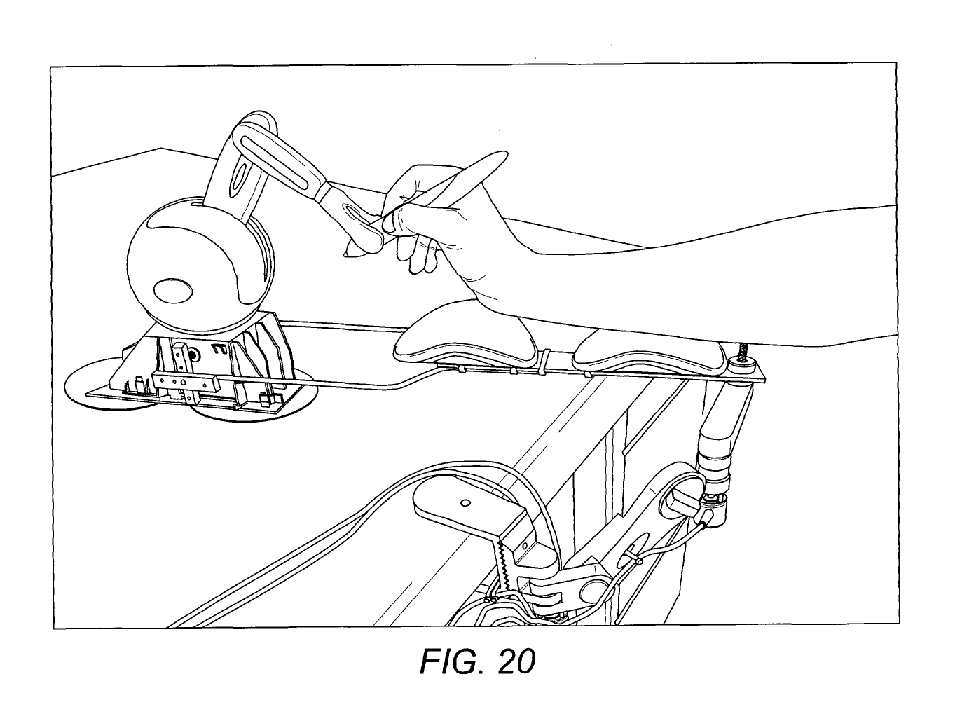

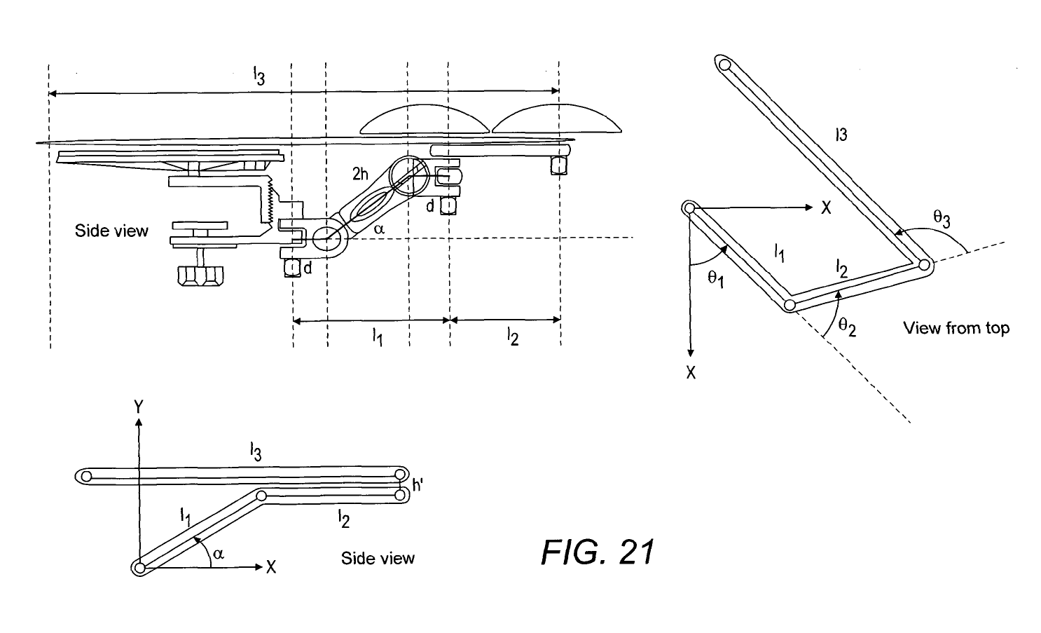

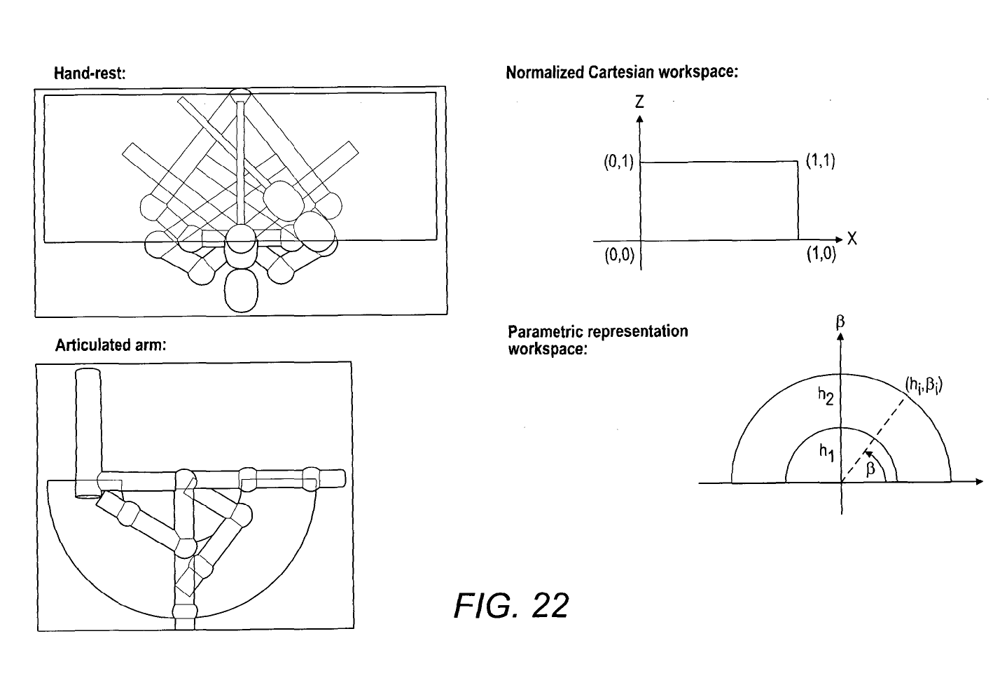

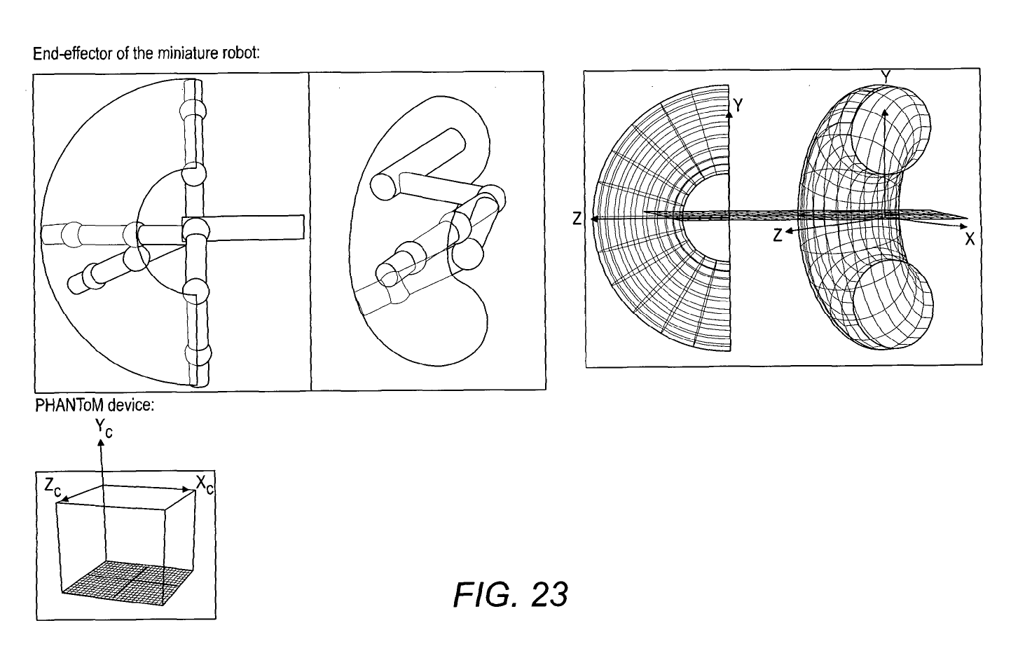

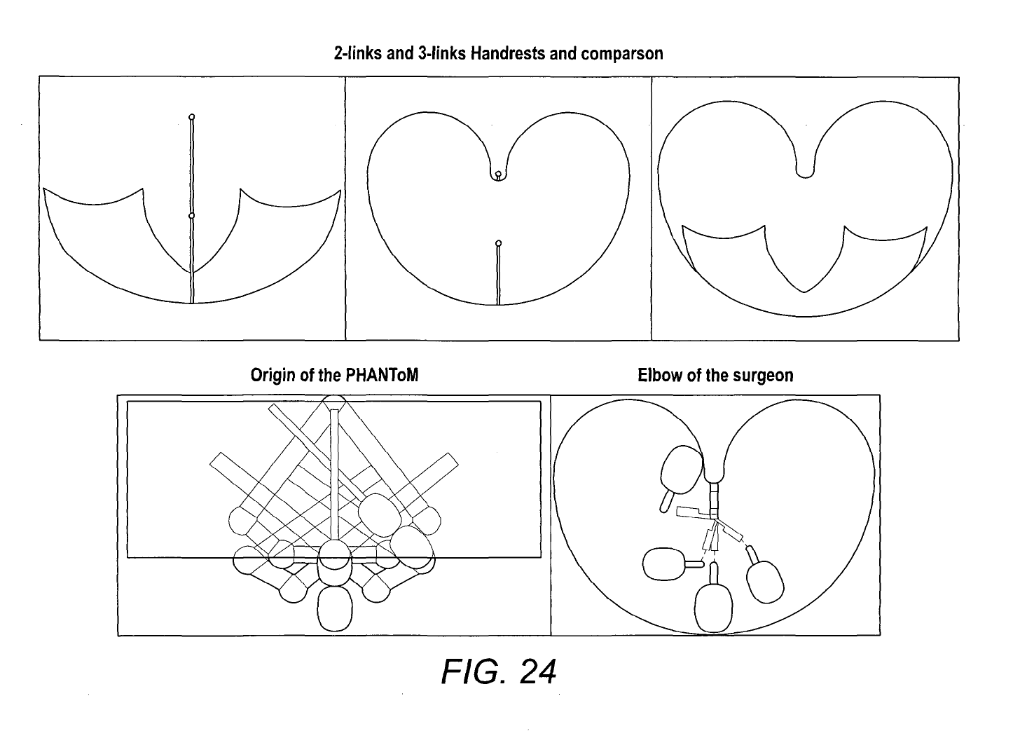

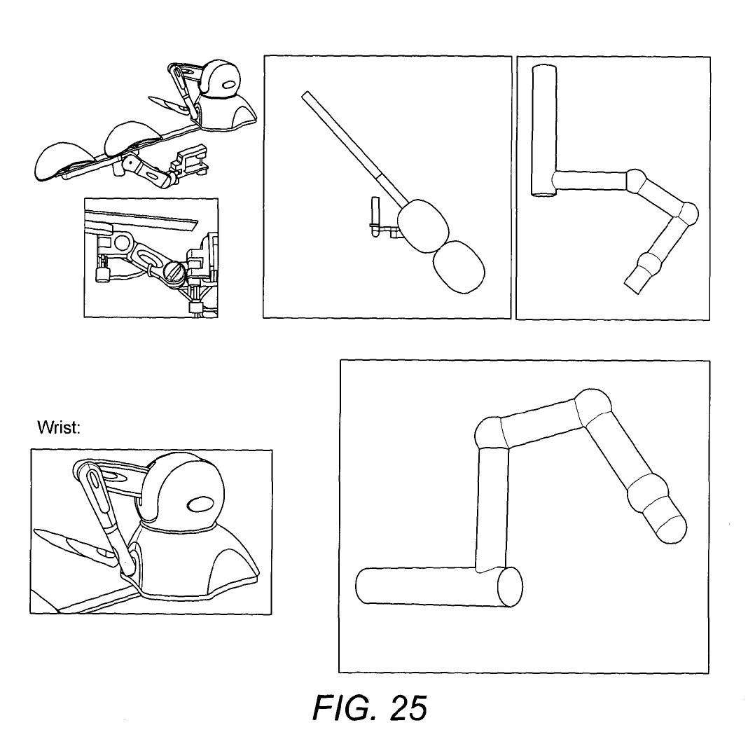

FIG. 2 shows an example embodiment of the support for carrying a haptic manipulator.

In FIG. 1, a haptic manipulator (110) is shown. The haptic manipulator (110) has first and second arms (112, 114), the first of which (112) is rotatable relative to a base of the haptic manipulator (110), and the second of which (114) is rotatable relative to the first arm (112). The haptic manipulator (110) further comprises a stylus element (116) which is rotatable and twistable with respect to the second arm (114). In use, the manipulator (110) sits upon a platform (118) and a user (120) rests their forearm on a forearm rest (122) and manipulates the stylus (116) so as to bring about movement of an end effector of a robot.

To provide the user with additional degrees of freedom for providing additional instructive information to the system for manipulating the end effector, the forearm support (122) is itself movable with respect to a surface (124). The forearm support (122) is, in this embodiment, coupled to the surface (124) by a linkage (126, 128, 130, 131) having first, second, third, fourth and fifth mechanical joints (132, 134, 136, 138, 139). A user with their forearm resting upon the forearm support (122) may move the forearm support (122) by the movement of their arm. The forearm support (122) may accordingly be considered to be an extension of the user's arm—like an exoskeleton. The additional degrees of freedom provided by the joints (132, 134, 136, 138, 139) enhance the range of motion (with respect to the surface (124)) that the user has available to them for providing instructions to the described system.

The platform (118) is fixedly coupled to the forearm support (122) so that movement of the forearm support also causes movement of the haptic manipulator (110). By maintaining a fixed relationship between the forearm support (122) and the haptic manipulator (110), the user is able to effect detailed and steady movement of the end effector over a large range of positions in a manner that is sympathetic to the movements that the user would perform if they were themselves holding the end effector.

The platform (118) not only supports the haptic manipulator (110), but also features a movement facilitating device (140), in this case an air cushion providing device, to reduce the friction between the platform (118) and the surface (124). By reducing the friction between the platform (118) and the surface (124), the user is able to move the forearm support (122) in a manner that closely follows the routine movement of their arm as, in routine movement of their arm through free space, the user would not experience any substantial impediment to the motion of their arm. The presence of an air cushion between the platform (118) and the surface (124) substantially eliminates friction therebetween thereby allowing the user easily to easily manoeuvre the forearm support (122) whilst reducing any sensation of drag that would otherwise have been caused by friction.

FIG. 2 shows, in greater detail, the forearm support (122) and the haptic manipulator support platform (118). As can be seen, the forearm support (122) is coupled to the platform (118) by a pair of rods (210) so as to keep the spatial relationship between the platform (118) and the forearm support (122) fixed. In this case, the movement facilitating member comprises a pair of circular elements (212) having, at their respective centres, tube coupling members (214) arranged to provide air to the under side of the circular elements (212) by way of flexible tubes (316). Flexible tubes (216) may be connected to a pump (not shown) so as to force air from the pump through the tubes to the circular elements (212) thereby providing an air cushion interposed between the circular elements (212) and the surface (124). A person skilled in the art will appreciate that the tubes need not be flexible and that air could instead be provided to the circular elements (212) by other means. Tubes (316) may, additionally or alternatively, be coupled to a vacuum so as to suck air from the underside of circular elements (212) to the vacuum source thereby causing suction between the platform (118) and the surface (124) so as to impede movement of the forearm support and platform assembly with respect to the surface (124).

A close up of the movement facilitating member can be seen on page AO of the appended annex. In that embodiment the circular elements comprise compact discs. A person skilled in the art would recognise alternative elements that could be used in order to impede or facilitate movement between a platform and a surface,

Advantageously, by providing a forearm support, tremors in the user's movements caused by movement between their arm and body are reduced. Furthermore, by supporting the user's forearm, user fatigue is advantageously reduced.

By supporting the user's hand and substantially removing friction between the platform and the surface, the precision of instructions that the user provides to the system may advantageously be improved.

By providing an air cushion on which the support platform sits, advantageously both static and dynamic friction are reduced.

As one alternative, the apparatus described herein may be employed to provide a user with further haptic feedback, for example, the movement facilitating member may be arranged so as to reduce, or remove, the air cushion between the haptic manipulator support and the surface thereby reintroducing some, or all, of the friction therebetween—thereby providing a mechanism for providing further haptic feedback to the user. This may be achieved, for example, by controlling the volume, or pressure of air feed to the movement facilitating member(s). As an alternative, a vacuum may be applied to the tubes and the strength of the vacuum used to simulate static and/or dynamic friction gradients.

As one possibility, the apparatus described herein may be controlled by a controlling device that has been pre-programmed to enforce boundary conditions to impede movement of the end effector into prohibited areas. For example, if the device described herein were to be used during surgery and, following a preoperative scan, a clinician identified an area of tissue that it would be desirable for the end effector to be kept away from, information regarding this could be provided to the system so that, during surgery, when the forearm support was moved with respect to the surface so as to bring the end effector within a given range of the identified area, a boundary condition could be enforced by the movement facilitating member, for example by providing negative pressure to the movement facilitating member through the tubes, so as to impede or prevent movement towards or over said boundary.

Advantageously the “hovercraft” style platform described herein may be used on a plurality of different surface types, including liquids.

A person skilled in the art will recognise that the device described herein is able to support, upon the platform, a range of different haptic manipulators and need not be limited to supporting a PHANTOM OMNI device.

Advantageously, the device described herein, does not require any motors to provide braking of relative movement of the component parts. Accordingly, the device is smaller than would be necessary if motors were employed, consumes less power, and causes substantially less local heating.

Annex page A4 shows a view of an example embodiment of the device described herein and shows, from the left, a disc clamp for clamping the linkage to a desk, a linkage, comprising a plurality of rotational joints each having a rotational encoder arranged to provide a computer with information for determining the location of the hand rest, a pair of hand rests for supporting a forearm of a user, and a haptic device—in this example a PHANTOM OMNI device. The haptic device is supported on a platform which comprises a pair of air cushion creating devices. In this example, the air cushion creating devices are formed from compact disc having hose pipes attached to their respective central holes.

Advantageously, by coupling the haptic manipulator to the forearm support, the effective range of the device described herein is substantially increased when compared to a haptic manipulator that is fixed relative to the surface and manipulated by a user employing an arm support that is moveable relative to the table. Further advantageously, this enables the employment of small haptic manipulators as, when mounted on the platform, the haptic manipulator only needs to be able to detect movement of the stylus over the limited movement range of the user's wrist and digits; if the haptic manipulator were fixedly coupled to the work surface, it would need to be able detect movement over a range equivalent to the sum allowable wrist, digit, and arm movements.

A person skilled in the art will appreciate that the haptic manipulator support platform and movement facilitating member combination may be made and sold separately to the rest and linkage combination.

A person skilled in the art will appreciate that each of the embodiments described herein are exemplary and that one or more features of any of the disclosed embodiments could be combined with one or more other features of any of the other embodiments disclosed herein. Appended annex pages A-2 to A12 describe exemplary and non-limiting embodiments.

### Overall Design

The proposed design allows users to comfortably adjust the position of their arm and control a haptic manipulator. All hand motions, including those controlling the haptic manipulator stylus, are translated into commands that control a robotic instrument similar to the ones mentioned above. The haptic manipulator is positioned on top of a “hovercraft” type platform. This allows frictionless steering of the haptic manipulator according to the forearm position of the user. Superimposed on top of the gross positioning of the manipulator, a further 6 degrees of freedom are provided by means of a stylus.

1-9: (canceled)

10: An apparatus comprising:
a platform for supporting a haptic manipulator device and comprising a movement facilitating member for supporting the platform on a surface;
a linkage; and
a rest fixedly coupled to the platform and arranged to be movably coupled to the surface by way of the linkage;
wherein the movement facilitating member is operable, when the rest is coupled to the surface, to change the amount of force required to move the rest relative to the surface.

11: The apparatus of claim 10, wherein the movement facilitating member is operable to reduce the amount of force required to move the rest relative to the surface.

12: The apparatus of claim 10, wherein the movement facilitating member is operable to reduce the amount of force required to move the rest relative to the surface by reducing friction between the platform and the surface.

13: The apparatus of claim 12, wherein the movement facilitating member is operable to reduce the amount of friction between the platform and the surface by providing an air cushion between the platform and the surface.

14: The apparatus of claim 10, wherein the movement facilitating member is operable to impede movement of the rest with respect to the surface, by suction.

15: The apparatus of claim 10, wherein the rest is an arm, hand, or wrist rest.

16: The apparatus of claim 10, further comprising a haptic manipulator device supported by the platform.
